(19) United States
(12) Patent Application Publication
(10) Pub. No.: US 2007/0167809 A1
(43) Pub. Date: Jul. 19, 2007

(54) Method and System For Estimating Cardiac Ejection Volume And Placing Pacemaker Electrodes Using Speckle Tracking

(75) Inventor: Praveen Dala-Krishna, Sicklerville, NJ (US)

Correspondence Address: HANSEN HUANG TECHNOLOGY LAW GROUP, LLP, 1725 EYE STREET, NW, SUITE 300, WASHINGTON, DC, 20006, US

(73) Assignee: EP MEDSYSTEMS, INC., West Berlin, NJ (US)

(21) Appl. No.: 11/610,888

(22) Filed: Dec. 14, 2006

Related U.S. Application Data
(63) Continuation-in-part of application No. 11/428,517, filed on Jul. 3, 2006.
Application No. 11/428,517 is a continuation of application No. 10/620,517, filed on Jul. 16, 2003, now Pat. No. 7,211,045.
(60) Provisional application No. 60/397,653, filed on Jul. 22, 2002.

(51) Int. Cl. A61B 8/14

(52) U.S. Cl. 600/459

(57) ABSTRACT

A method and system for estimating the volume of blood ejected from a cardiac ventricle or atrium uses ultrasound to track speckle patterns in the heart. The process utilizes the M-mode to estimate volume differences in a view of the ventricle or atrium over time using speckle pattern motion to estimate volume differences between systole and diastole. Alternatively, the ultrasound speckle tracking method may be used in combination with Doppler processing techniques or to obtain temporal flow profiles across flow cross-sectional areas, from which the flow volume is computed. The method can also measure the phase delay of motion of sites on the cardiac wall relative to each other or relative to an ECG signal.

### RELATED APPLICATIONS

This application is a continuation-in-part of U.S. patent application Ser. No. 11/428,517 filed Jul. 3, 2006, which is a continuation of U.S. patent application Ser. No. 10/620,517 filed Jul. 16, 2003 now U.S. Pat. No. ______, which claims the benefit of priority to U.S. Provisional Application Ser. No. 60/397,653, filed on Jul. 22, 2002, the entire contents of all of which previous applications are hereby incorporated by reference.

### FIELD OF THE INVENTION

The present invention relates to a technique for measuring cardiac output (i.e., total volume of blood ejected by the left ventricle in one cardiac cycle) using ultrasonic imaging, particularly for use in evaluating placement of cardiac pacing electrodes.

### BACKGROUND OF THE INVENTION

Volumetric output of blood from the heart and/or circulatory system is of interest in various diagnostic and therapeutic procedures. Such measurements are of significant interest during electrophysiological evaluation/therapy to first evaluate the extent of dysfunction due to arrhythmia and subsequently to judge the effect/effectiveness of any ablations/therapeutic procedures that are carried out on the cardiac muscle/conduction system. Iwa et al., Eur. J. Cardithorac. Surg., 5, 191-197 (1991).

Ultrasound is the imaging modality of choice, especially in cardiology, since this modality offers real-time imaging capabilities of the moving heart. Further, advances through Doppler techniques allow the physician to visualize as well as measure blood flow. Pulse wave and continuous wave Doppler have proven to be quite accurate, and an effective way of evaluating flow through various parts of the circulatory system, especially the heart. Tortoli et al., Ultrasound Med. Bio., 28, 249-257 (2002); Mohan et al., Pediatr. Cardiol. 23, 58-61 (2002); Ogawa et al., J. Vasc. Surg., 35, 527-531 (2002); Pislaru et al., J. Am. Coll. Cardiol., 38, 1748-1756 (2001).

Other technologies, including washout curves of contrast agents have been proposed to measure flow volume, especially to compensate for loss of signal quality due to imaging depth. Krishna et al., Ultrasound Med. Bio., 23, 453-459 (1997); Schrope et al., Ultrasound Med. Bio., 19, 567-579 (1993).

However, until recent advances in miniaturized ultrasonic transducers, physicians were limited to only certain angles of view, thus limiting the range and effectiveness of possible measurements. Further, given the depth of imaging required by such classical approaches, associated interrogation frequency limitations due to attenuation restricted the accuracy of measurement. Krishna et al., Phys. Med. Biol., 44, 681-694 (1999). With the recent introduction of catheter based transducers for imaging the heart from either the vena-cava or even from within the heart, such limitations on frequency of interrogation and angle of view are not applicable.

One specific need for this invention is for the permanent placement of cardiac pacing electrodes. Cardiac pacing has been around for many years, and essentially involves the placement of a permanent electrode in the right ventricle to quicken the pace of an otherwise slow heart. A new therapy has recently been introduced to the market, which involves pacing of the left ventricle in conjunction with the right ventricle in an effort to “resynchronize” the heart, that is, to coordinate the left ventricle's contraction in time with the contraction of the right ventricle. One problem in the current therapy is the optimization of the placement of the left ventricular electrode so as to provide maximum therapy. This invention addresses this problem by providing intracardiac ultrasound imaging and ultrasound Doppler as a new tool in the placement of the electrode.

### SUMMARY OF THE INVENTION

An embodiment tracks ultrasound “speckle” reflections to accurately measure ventricle volume and changes in volume using multiple M-mode measurements. Such an M-mode based embodiment can include hardware and/or software, either on the ultrasound system, or on a separate system that directly or indirectly communicates with or receives data from the ultrasound system and a device that can digitize and/or transmit ECG data, if separate from the ultrasound unit. This device can utilize ultrasound data, in coordination with the ECG signals, to calculate the spacing between the walls of the left ventricle to estimate the maximum and minimum volumes of the ventricle in the course of a cardiac cycle.

By combining the ultrasound system with a robust cardiac electrophysiology recording device such that both surface electrocardiograms and internal electrocardiograms can be recorded and displayed. Both electrocardiograms, while not necessary, may assist in the procedure. In addition, combining a pacemaker programmer with a comprehensive electrophysiology recording device could eliminate errors of input, reduce duplication of demographic data, and allow all data to be recorded in one database at one time. Also, by implanting an electrode in a spot chosen by imaging as well as voltage mapping, an overlay of these two parameters could more easily allow the physician to visualize the mechanical and electrical characteristics at the same time. Moreover, by automatically or semi-automatically designating one or more M-mode lines to be used to track points on the atrial or ventricular walls the system can estimate the volume of the chamber at a specific instant in the cardiac cycle. Further, by tracking, on an image-to-image basis, the location and motion of local regions of ultrasound speckle in the sequence of images of the cardiac muscle wall or in the sequence of images of the flow of blood through the heart can be used to estimate the volume of cardiac output. The location and motion can also be used to correlate the local motion with electrocardiogram data or with the motion of other regions to determine how well the local region is synchronized (or delayed) with respect to the cardiac cycle. Still further, by integrating a cardioversion device or defibrillation electrode with the ultrasound catheter.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain features of the invention.

FIG. 1 provides a general system diagram showing an ultrasound system.

FIGS. 2A, 2B, and 2C illustrate various embodiments of the present system with an attached workstation.

FIG. 3 illustrates a typical B-mode image and an associated Doppler spectrum. A cross-sectional view of the ventricle and the aortic valve are shown as viewed from the right atrium. The spectral Doppler waveform shows the velocity profile of the flow at the aortic valve.

FIG. 4 illustrates the basic technique to measure volume of flow from a spectral Doppler spectrum, and the approximate correlation of the ECG with the Doppler spectrum readout.

FIG. 5A and FIG. 5B illustrate the measurement technique for calculating cross-sectional area of the output from the ventricle. In this view, the ultrasound catheter is positioned in the vena-cava or in the right atrium.

FIG. 6 illustrates the basis of Doppler measurement used in an embodiment by delineating streamlined flow through a vessel, its profile through time and the basis of the time-integral area product showing volume of flow.

FIG. 7A-7D illustrate the basis of ejection volume measurements according to various embodiments.

FIG. 8 provides a perspective view of an ultrasound system for use in the present invention.

FIG. 9 illustrates a normal heart (i.e., non-congestive failure (CHF) heart). Panel A illustrates the right atrium (RA), left atrium (LA), right ventricle (RV), and left ventricle (LV) as well as the location of an electrode (“lead”) placed on the right ventricle to provide electrical pulses to the heart; the directions of the normal pacing pathways are also shown. Panel B illustrates the direction of normal contraction of the heart muscle in the ventricles.

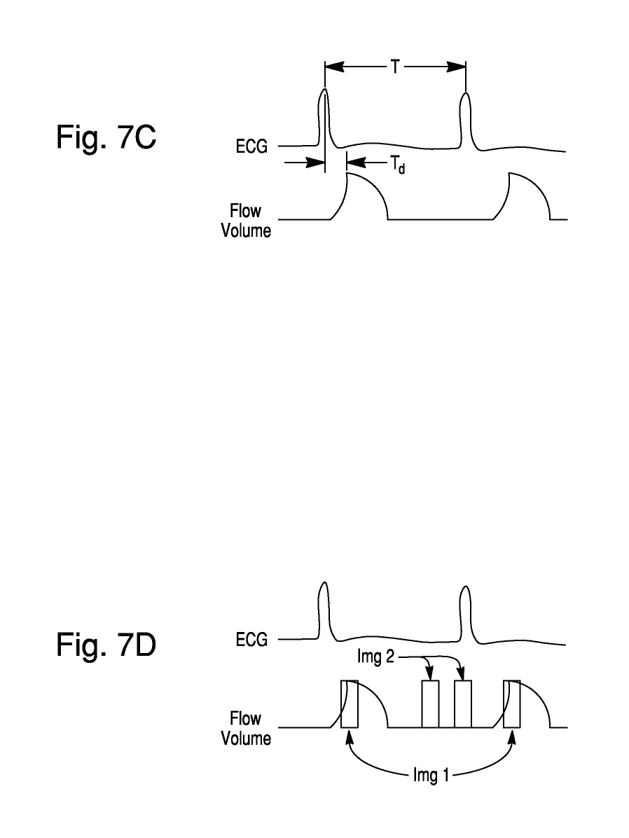

FIG. 10 illustrates a CHF heart with enlargement of the left ventricle. Panel A illustrates the enlargement of the left ventricle normally observed with CHF; the dotted line in the left ventricle is included to illustrate the normal heart (i.e., non-CHF) as shown in FIG. 9. Panel B generally illustrates the area slow conduction and the normal area for placement of an electrode for re-synchronization. Panel C generally illustrates the direction of potential contraction normally associated with CHF without re-synchronization.

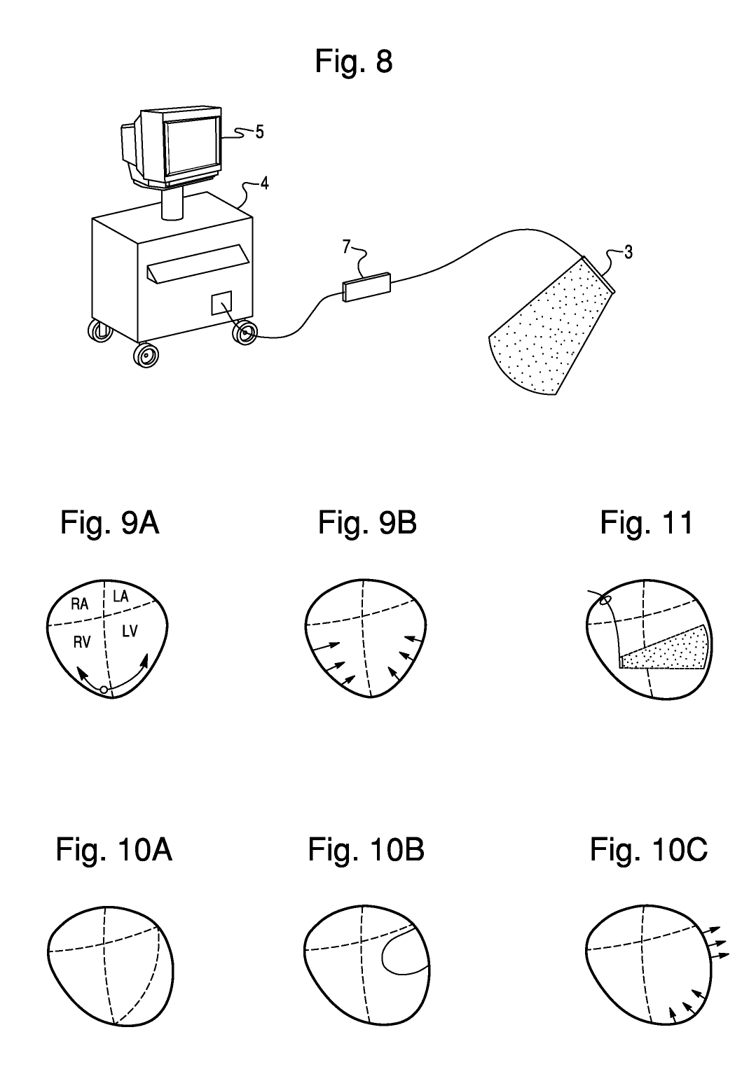

FIG. 11 illustrates placement of the ultrasound catheter in the right ventricle to image the left ventricle according to one embodiment of the present invention.

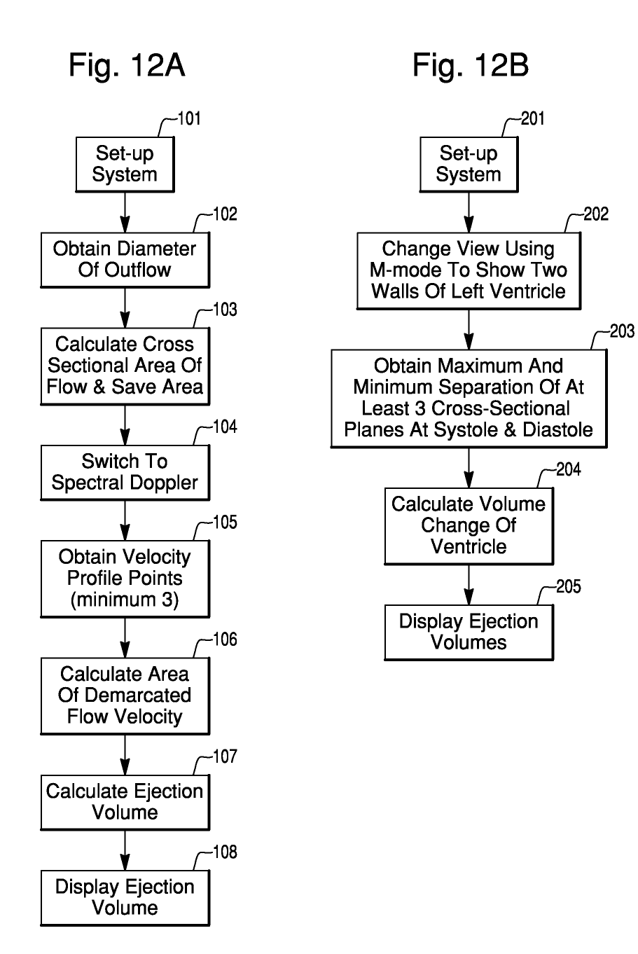

FIGS. 12A through 12D provide computer flowcharts illustrating procedures for estimating cardiac output.

FIG. 13 illustrates an example of a display showing motion paths of tracked local regions of ultrasound speckle.

FIG. 14 provides a flowchart illustrating a procedure for determining a potential location for a pacemaker implant.

### DETAILED DESCRIPTION OF THE PREFERRED EMBODIMENT

Heart failure is a disease where the heart's main function, a pump for blood, is wearing down. The heart tissue can absorb fluid, the left ventricle does not allow quick electrical conduction, becomes enlarged, does not contract well, and becomes less efficient at pumping blood. A measurement for the cardiac output (volume of ejected blood) is called the “ejection volume”. The efficiency of the heart as a pump is called the “ejection fraction” or “EF”. EF is measured as the percentage of the blood volume contained in the ventricles that is pumped out with each beat of the heart. A healthy, young heart will have an EF greater than 90 (i.e., 90 percent of the ventricular blood is pumped with each heart beat); an older, sick heart in heart failure can have an EF less than 30. Heart failure leads to an extremely diminished lifestyle, and, left untreated, can be a major cause of mortality.

A new therapy to treat heart failure is bi-ventricular pacing, or “resynchronization” therapy, where both ventricles of the heart are paced with an implantable pulse generator, commonly known as an artificial pacemaker. Normal pacing for a slow heart is performed via an implanted electrode in the right ventricle. The conduction myofibers (Purkinje fibers) conduct the electrical pulse and the ventricles contract synchronously in an inward direction, resulting in blood being pumped efficiently from the heart. In heart failure, the left ventricle becomes enlarged and conduction through the tissue of the left ventricular wall often becomes slow, so that the upper part of the left ventricle conducts as much as 200 to 250 milliseconds behind the apex area of the ventricles. This leads to poor and uncoordinated contraction, and in many cases, an outward movement of the heart muscle, so that blood sloshes around rather than being squeezed out of the ventricle. Thus, an ideal location to place a pacing electrode in the left ventricle is in the area of slowest conduction, which can be a rather large area of the left ventricle, and may not always be the area that has the largest conduction. The problem facing physicians today is to locate the optimal spot for the permanent fixation of the pacing electrode. The thrust of this invention is to provide a method and device to optimize the location of the electrode.

As used herein, the term “site” generally refers to a specific physical or anatomical feature or location on or within the heart, regardless of where location or feature moves spatially over time. The term “image location” generally refers to where an anatomical site is located within an ultrasound image at a specific time. An image location may be defined by 2-D pixel coordinates or by coordinates within an external frame of reference and the moment of time within a cardiac cycle. The term “3-D location” generally refers to where in a spatial coordinate system an anatomical site is located at a specific moment of time. A 3-D location may be located within by 3-D coordinates (e.g., X,Y,Z) and the moment of time. The term “point” may be used interchangeably with “location”. Nevertheless, the image location in a specific ultrasound image may correspond to a specific anatomical site, which in turn is located at a specific 3-D location at the moment corresponding to the image. Therefore, when describing a specific ultrasound image, the terms are often blurred, and a term sometimes may infer the physical site, the site's 3-D location at the moment of image capture, and/or the site's image location at the moment. The term “position” and its related forms usually include both the concept of a 3-D location and the concept of a 3-D orientation.

A standard pacemaker electrode is ideally implanted at a site in the cardiac wall which achieves the lowest “threshold.” That is to say the site for which the lowest voltage level is needed to excite the surrounding tissue to conduct synchronously the pacing signal from the electrode. Thus, the electrode is implanted based upon merely finding the site where the lowest voltage is needed to “capture” the tissue. Placing the pacemaker electrode in the optimal site is not an easy task. Ideally, a site is chosen which optimizes the EF. Finding a site with a low threshold, while desirable, is not as important as optimizing EF. Thus, the ability to not only visualize the motion of the left ventricular wall, but also measure EF, or some form of output of the heart, such as stroke volume or flow rate, is highly desirable during the implantation procedure. The various embodiments use ultrasound technology to provide this ability.

The present invention is directed to methods and systems for measuring volumetric flow, specifically cardiac output, either with minimal intervention/input from the physician, or automatically, with the user of the system pre-specifying certain operating parameters or measurement criteria. One embodiment of the present invention is in the form of hardware and/or software that exists as part of the ultrasound scanner. In such an embodiment, the system utilizes the Doppler processing capabilities of the host ultrasound scanner to obtain a time-varying signal representative of the velocity of flow through an area of interest. Such area could include the inlet of the aorta from the left ventricle, or the valve in between. The system also utilizes a view or measure of the cross-sectional area through which the flow of interest is to pass.

The Doppler signals are utilized by the processor, or any other hardware, software, or combination thereof, to calculate volume of flow through the area of interest. Boundaries for the Doppler signals can be either demarcated by the user, or automatically estimated by the system. Also, the measure of the cross-sectional area through which such flow passes can be either demarcated/input by the user, or can be automatically measured by the system,

Other embodiments also include the measuring system, either in the form of software and hardware or a combination thereof on a separate workstation/computer that is capable of obtaining relevant data from the examining ultrasound scanner either directly or indirectly, and methods of triggering or correlating the ultrasonic/Doppler signals (video/audio) with the electrocardiogram (ECG) of the subject being examined.

Another embodiment utilizes the Doppler audio output of the Doppler processing system/sub-system in the ultrasound machine in addition to the facilities to obtain the measure of the area of interest through which the flow is to pass, and the ECG of the subject being examined. Again, this process/system can be embodied within the hardware and/or software of the ultrasound scanner, or implemented as a workstation and/or computer separate from the ultrasound scanner with facilities to communicate either directly or indirectly with the ultrasound scanner. Such processing then uses the frequency, phase, and amplitude of the audio signals, along with the measure of the area of interest through which the flow exists, to calculate the volume of flow.

A further embodiment can also include methods of obtaining electrocardiogram (ECG) data from the subject being scanned to enhance the demarcation and/or separation of signals from beat to beat of the heart or to assess, either automatically or aided by a user, the condition of the cardiac system and hence the factors effecting the acquired Doppler data.

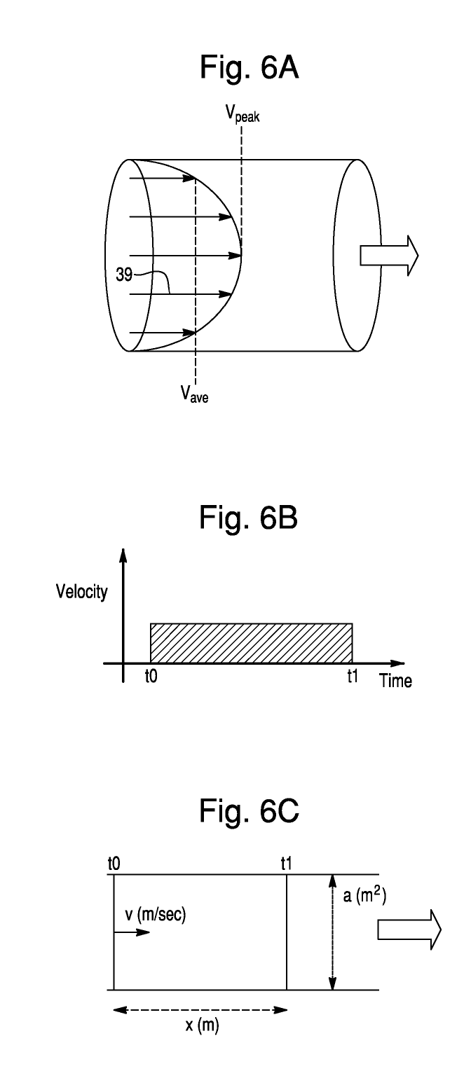

Further, an embodiment may provide a manual control or an automated detection of the delay T_{d} between the ECG R wave and the maximum measured ventricular volume, and possibly also the delay between the ECG R wave and the minimum ventricular volume. This may be of particular significance for pacemaker configuration and electrode placement. This delay is illustrated in FIG. 7B. It is noted that the delay T_{d} may vary from cardiac cycle to cardiac cycle, especially when pacing stimulation is applied, so measurement of the delay T_{d} may need to be made at various points throughout a procedure.

Ultrasound, as an imaging tool, has been around for some time. However, imaging through the chest is very difficult in that the ribs block the view and that the depth of penetration gives poor resolution. Ideally, the ultrasound transducer should be positioned closer to the heart. An esophageal ultrasound probe has been used on many patients in an attempt to view the heart. See, e.g., Jan et. al., Cardiovasc. Intervent. Radiol., 24, 84-89 (2001). Unfortunately, the results may be less than desired since the probe must view through the esophagus and both walls of the heart, lending to less resolution in the image than desired. Intravascular ultrasound systems, although ideal in its size with thin catheters, generally utilize high frequencies which result in poor depth of penetration. X-ray or X-ray fluoroscopy may give good images of the electrode, but not of the actual tissue of the heart (most particularly the walls of the ventricle).

The present invention overcomes these problems. Preferably, the present invention uses an ultrasound imaging catheter designed for intracardiac use. Such an intracardiac catheter is generally sized as 10 French or less, has multiple elements on the transducer (e.g., 48 or 64 elements), employs lower frequencies (e.g., about 5 to about 10 MHz), and uses a phased array transducer for optimal resolution. Not only will this allow the imaging of wall motion for this specific purpose of a left ventricular electrode fixation, but will also, especially if used in conjunction with Doppler techniques, provide information to calculate measurement of cardiac output.

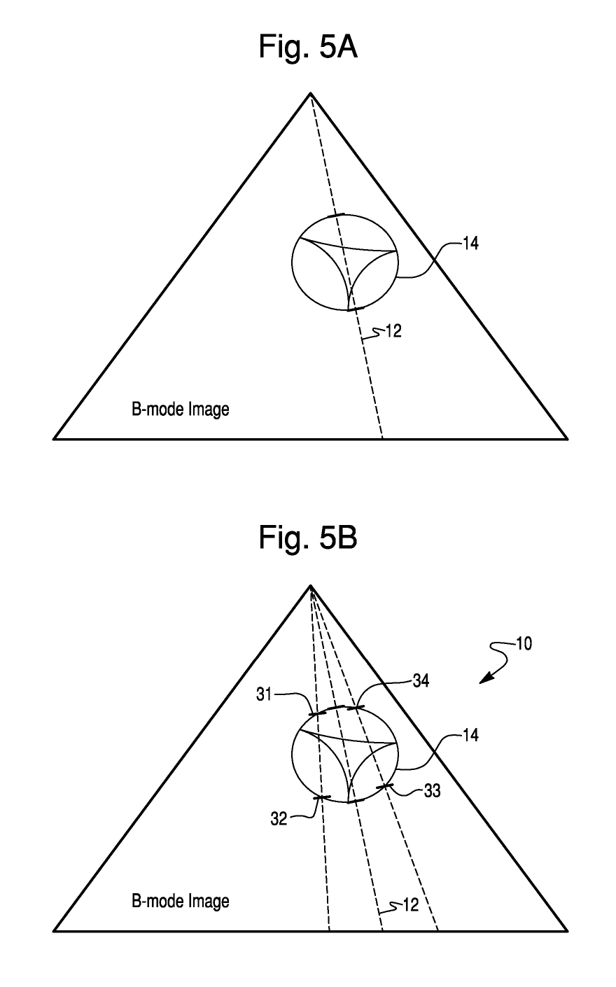

Such a catheter could be placed in either the right atrium of the heart or the right ventricle and easily allow viewing of the left ventricle as shown in FIG. 7. Another approach for viewing could be from the outside of the heart, via an incision through the chest of a patient. This catheter would connect either directly to a display system or through a connecting cable, as shown in FIG. 6. The ultrasound display can provide a display of the measurement of cardiac output in assisting the physician with the procedure.

In addition to ultrasound imaging, a number of other items may make this implant an easier procedure, especially since many of the heart failure physicians may not have previously implanted pacemakers, may not have access to x-ray fluoroscopy, may have limited budgets for capital equipment, and may desire all discreet components used in an implantation to be accessible through one keyboard, allowing for better patient data management.

Often times the heart failure patient has a number of co-morbidities showing symptoms at the same time, such as atrial fibrillation, ventricular tachycardias, and renal failures, among others. Atrial fibrillation and ventricular tachycardia can be brought under control via electrical shock cardioversion, either internally with catheters, or externally, although with much higher energy, with patches or paddles. A cardioversion device which could utilize the same electrodes that are otherwise introduced into the heart for pacemaker implantation, would be advantageous if also integrated with the overall electrophysiology system. In this manner, inadvertent shocks could be avoided as the trigger mechanism would come from the ventricular signal from the internal electrode. Thus, in an embodiment, the ultrasound imaging system of the present invention also comprises an integral defibrillation system whereby, if needed, internal cardiac defibrillation can be implemented quickly and easily. The integrated defibrillation electrode or system may be incorporated into the ultrasound imaging catheter, attached to the ultrasound imaging catheter, or as a separate electrode system or catheter which is inserted along with the ultrasound imaging catheter.

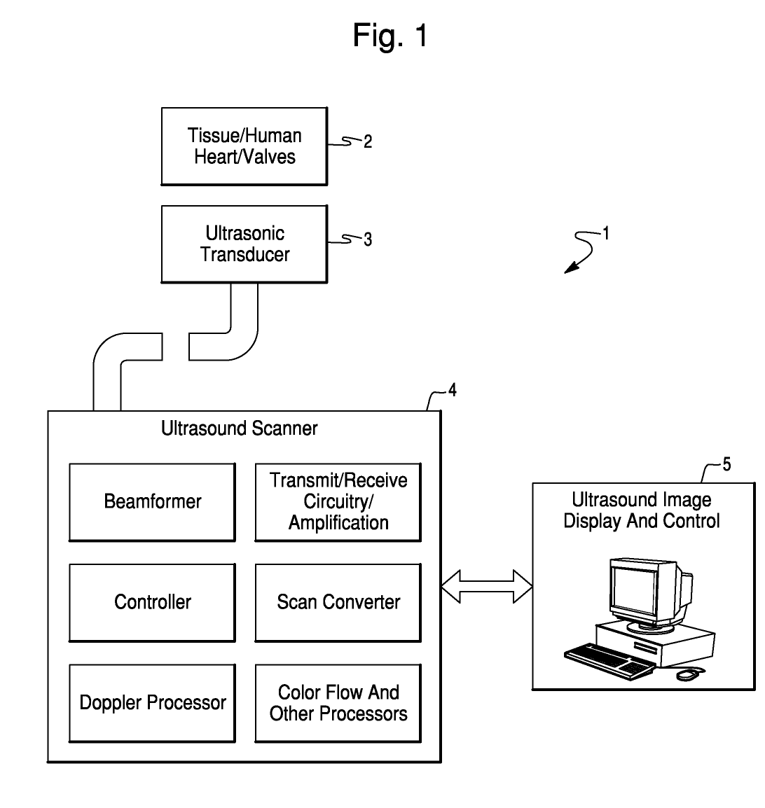

A diagram of an embodiment of the present invention is shown in FIG. 1. As shown in FIG. 1, an ultrasound imaging system 1 suitable for measuring cardiac output of a patient's heart 2 may include an ultrasound imaging catheter which houses at least one ultrasonic transducer 3 which utilizes piezoelectric properties to generate acoustic signals from electrical signals in order to obtain ultrasound signals. The ultrasound transducer 3 is of a size and type suitable for insertion into the patient's heart and is used to obtain ultrasound signals associated with an area of the patent's heart in which cardiac output is to be measured. The signals received from the ultrasound transducer 3 are fed into an ultrasonic scanner unit 4 which contains the necessary digital and/or analog electronics to generate and process ultrasound signals from the at least one ultrasonic transducer 3 to generate B-mode, M-mode, or Doppler representations of the patient's heart. These digital and/or analog electronics may include, for example, a beamformer, transmit/receive circuitry and amplification circuitry, a controller unit, a scan converter, a Doppler processor and color flow as well as other processors. In addition, the system includes an associated computer 5 that can generate and process the ultrasound signals in order to measure the cardiac output in the patient's heart and to measure the delay in the motion of a site on the cardiac wall with respect to the cardiac cycle.

To utilize the system of the various embodiments, a clinician may face the need to place an electrode at a desired site at or near the left ventricle of a patient's heart in order to electrically activate the left ventricle of the patient's heart using the electrode. To achieve this placement at a desired site, the user would advance the electrode to the proximity of the upper left ventricle. Before or after the electrode is in the proximity of the upper left ventricle an ultrasound imaging catheter can be positioned to image the left ventricle of the patient's heart. The ultrasound imaging catheter houses at least one ultrasound transducer that utilizes piezoelectric properties to generate acoustic signals from electrical signals in order to obtain ultrasound signals. Moreover, the at least one ultrasound transducer is of a type suitable for insertion into the patient's heart and capable of obtaining ultrasound signals associated with an area of the patent's heart. Once the ultrasound imaging catheter is in place it is used to image the electrode at or near the left ventricle of a patient's heart and to guide the electrode to the desired site. Once the electrode is guided into the desired site the electrode is affixed to the heart. One desired site for attachment of the electrode is the upper portion of the left ventricle (i.e., nearer the base of the heart as compared to the apex).

In an embodiment, at least one transducer has a deflecting or rotation element whereby the transducer, once positioned to image the left ventricle of the patient's heart, can be easily rotated or moved in order to image other portions of the patient's heart.

Another method for placing an electrode at a desired site on a patient's heart can be implemented in order to electrically activate the left ventricle using the electrode. In accordance with this embodiment method the ultrasound imaging catheter is positioned to image a ventricle of the patient's heart. The ultrasound imaging catheter houses at least one ultrasonic transducer which utilizes piezoelectric properties to generate acoustic signals from electrical signals in order to obtain ultrasound signals. The at least one transducer is of the type which is suitable for insertion into the patient's heart and is capable of obtaining ultrasound signals associated with an area of the patent's heart. Once the ultrasonic imaging catheter is appropriately positioned to obtain a useful image, tracking the image location of at least one site on the ventricular wall is initiated to measure the motion of the site and determining the delay in motion relative to an electrocardiogram signal or relative to another site on the ventricle. This step of tracking the image location of at least one site on the ventricular wall is repeated to select another site of the ventricular wall until a site S of maximal motion delay is discovered. Once the site S is discovered, the electrode is affixed near site S.

The tracking step can be accomplished by tracking the changing image locations of the image of a site on one or more M-mode lines which are chosen to traverse the image of the ventricular wall. In an embodiment, the tracking may be accomplished by tracking, from one ultrasound image to the next, the motion of a site containing a “speckle” reflection or pattern. “Speckle” refers to a bright spot or spots of reflected ultrasound that typically appear in a B-mode ultrasound image of tissue. Without being limited to a particular theory of speckle formation, it is believed that local acoustic properties of tissue, such as thicknesses of tissue layers and changes in density or the speed of sound within tissue layers, lead to increased reflected sound energy. Providing a localized and “bright” spot on tissue, such speckle spots can be used according to various embodiments to track movements of particular sites on tissue walls.

In another embodiment of the present invention the system includes an ultrasound imaging system 1 to assist in cardiac electrophysiology procedures related to a patient's heart The system includes an ultrasound imaging catheter which houses a multi-element array transducer 3. The multi-element array utilizes piezoelectric properties to generate acoustic signals from electrical signals in order to obtain ultrasound signals. The multi-element array transducer is of a type suitable for insertion into the patient's heart and capable of obtaining ultrasound signals associated with the patent's heart. The system further includes an ultrasound scanner 4 which houses the necessary digital and/or analog electronics capable of generating and processing ultrasound signals from the multi-element array transducer to generate and display a representation of (a) the electrocardiogram of the patient's heart, (b) a real time image of the patient's heart, or (c) the cardiac output of the patient's heart. In an embodiment, the representation of ultrasound signals can be displayed relative to, and compared to, a voltage conduction map of the patient's heart (i.e., a representation of the progression of electrical activation/deactivation or “action potentials” of the muscles of the heart). These electronics may include, for example, a beamformer, transmit/receive circuitry and amplification circuitry, a controller unit, a scan converter, a Doppler processor and a color flow Doppler image generator, as well as other processors.

The basis of the measurement/estimation process of various embodiments of the present invention is shown in FIGS. 6A-C and 7A-D.

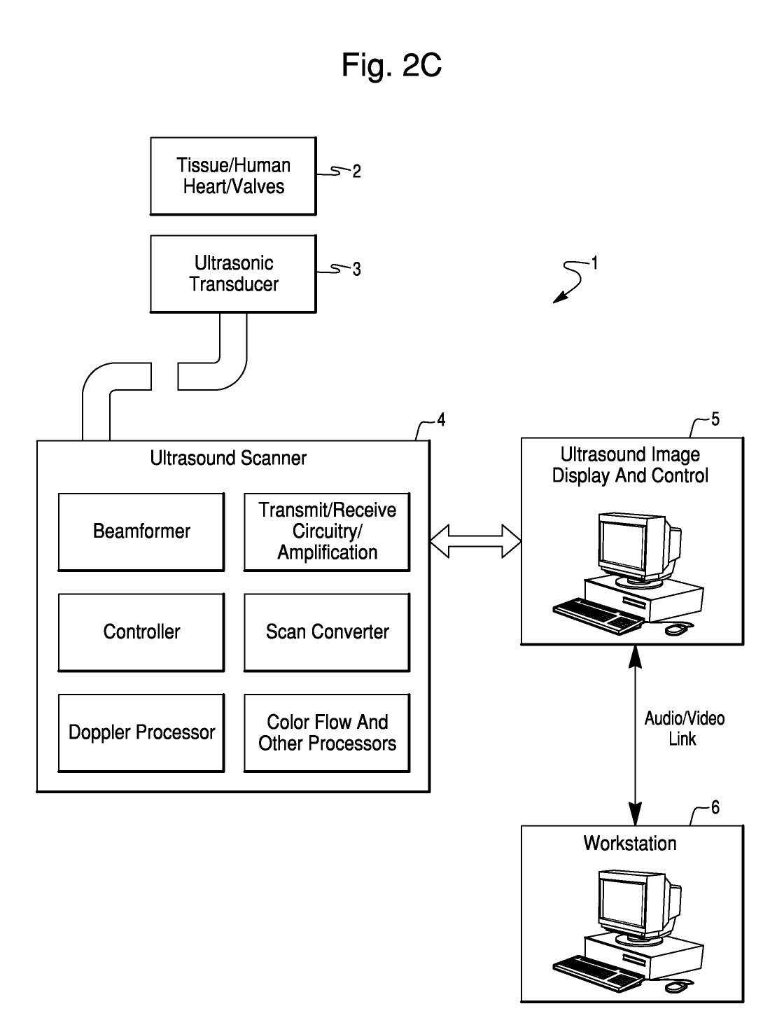

As shown in FIGS. 6A-6C, where the Doppler process is used the peak amplitude of the velocity profile is halved to provide the average velocity across the flow area (FIG. 6A). The velocity V is integrated (FIG. 6B) with respect to time from the start of the pulse (t0) to the end of the pulse (t1) yielding a distance x. Such integration can also include the negative peaks 22 shown in FIG. 4A to compensate for reverse flows. The resulting distance x of this integration with respect to time is then multiplied by the cross-sectional area A of the flow to provide the ejection volume (FIG. 6C). The integration length can also be set by integrating during the complete cardiac cycle (i.e., through one complete cycle of the ECG). The spectrum in FIG. 6 can also be obtained by either frequency and/or amplitude plotting of an audio signal.
Vₑⱼₜ=A∫Vₚₑₐₖ(t)/2 dt Eq. 1a
where: Vₑⱼₜ=ejection volume/stroke volume;

A=cross sectional area of flow; and

Vₚₑₐₖ(t)=peak velocity as a function of time t.

An alternative computation for Vₑⱼₜ is provided by Eq. 1b, which is amenable to M-mode ultrasound images of the heart obtained at discrete imaging intervals Δt as illustrated in FIG. 7A. In this computation, dimensional measurements of the vessel diameter obtained from M-mode images are used to estimate ejection volume.

$$\begin{matrix}{V}_{ejt} = {\sum }_{t = 0 … T} π {R}^{2} ( t ) L ( t ) \\ = {\sum }_{t = 0 … T} π {R}^{2} ( t ) v ( t ) Δ t\end{matrix} \qquad Eq . 1 b$$

where: Vₑⱼₜ=ejection volume or stroke volume;

R(t)=one-half the distance between chamber walls in the M-mode image;

L(t)=v(t) Δt=flow distance during time Δt time t; and

v(t)=mean flow velocity at time t.

FIG. 7B graphically illustrates the computation using Eq. 1b using a simplified rendering of an M-mode image. The value of R(t) may be measured manually on the ultrasound image display using calipers built into the M-mode ultrasound display or may be determined automatically using a computer graphics edge detection algorithm. As used herein, “calipers” refers to maximum and minimum measurements or threshold settings, which may be illustrated on a graphical display with horizontal indicators at the maximum and minimum levels or brackets. Using calipers, the maximum and minimum measured distance measurements or image locations of sites on the ventricle wall can be indicated with the upper and lower bounds of the caliper, so that the displacement distance is indicated by the separation between the end locations of the caliper. Because ultrasound can measure R(t) and dt accurately, the error in Vₑⱼₜ mainly depends on the accuracy of the measurement of v(t).

An alternative embodiment utilizes ultrasound speckle tracking to measure the flow velocity instead of utilizing Doppler techniques. The embodiment may determine the maximum flow rate and divide by two, as in Eq. 1a. Alternatively, the embodiment may make several flow velocity measurements 39 (as in FIG. 6A) throughout the cross-section of the flow area, compute the average of the measurements 39, integrate it over the time of a cardiac cycle, and multiply the average by the cross-sectional area to estimate Vₑⱼₜ.

Speckle pattern tracking has been previously studied, for example, as a possible alternative to Doppler flow measurement. See Ben A. Lin, Shmuel Einav, and Morteza Gharib; “Digital Ultrasound Speckle Image Velocimetry for Quantitative Cariovascular Flow Visualization”, 2003 Summer Bioengineering Conference; Jun. 25-29, 2003; Sonesta Beach Resort, Key Biscayne, Fla. Because speckle tracking is so computer processor time intensive, techniques such as fast Fourier transforms may be used to speed up cross-correlations of speckle patterns of two consecutive video frames. Speckle patterns can be tracked, because speckle patterns are relatively stable as they move—at least from one frame to the next. Note that speckle tracking can measure motion in any direction, not just along a selected M-mode line on a B-mode image. Normally ultrasound speckle is considered a detriment to visualization, so there has been much research into reducing speckle. Because reducing speckle can make features on the scale of the speckle disappear, any speckle reduction for viewing enhancement should be performed after analyzing the speckle pattern motion.

Using the M-mode process (FIG. 7A), the system outputs the relative image locations 41, 42 of sites on the two walls of the ventricle as a function of time. The ventricle can be approximated by an ellipsoid shape, whose secondary radius R₂ is represented by half the distance between the two wall sites 41, 42 measured by the M-mode, that is R₂=D₂/2. The distance D₂ may be measured using virtual “calipers” 43 on an M-mode which can be provided on the display of an ultrasound system. While some diagnostic procedures require obtaining an estimation of ventricle volume, others require only estimating a change in volume. Where a change in volume is desired, measuring a parameter related to volume, such as the diameter of a cross-section, may provide sufficient information to estimate the change.

The primary equation for the ventricle volume would therefore be
V=(4π/3)·(R₁+C₁)·R₂²+C₂ Eq. 2a
where: V=volume;

R₁=primary radius=length of the ventricle;

R₂=secondary radius=distance between the walls of the ventricle;

C₁=a correction factor to compensate for the difference in morphology of the ventricle with respect to an ellipse; and

C₂=a correction in the primary radius to compensate for longitudinal contractility of the ventricle during a cardiac cycle.

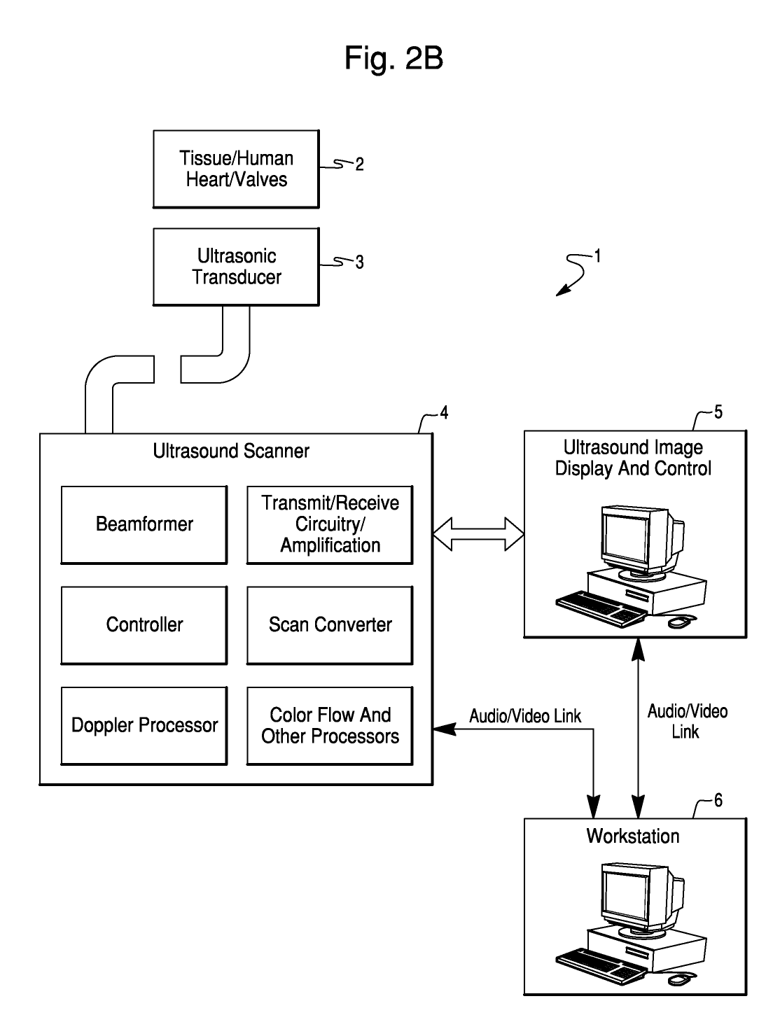

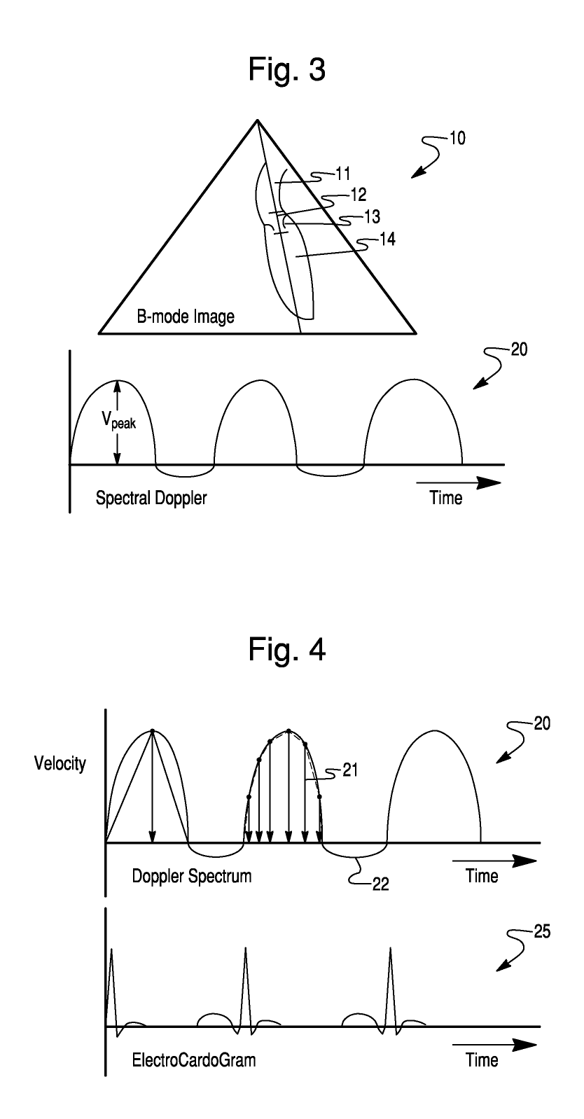

If more than one M-mode line 12 can be defined on a B-mode image 10 (FIGS. 3, 5A, 5B), then the current locations and motion of more than two sites on the ventricular wall can be tracked. Knowing the current locations of four or more sites 31, 32, 33, 34 on the ventricular wall at some moment allows a more accurate estimation for the ventricular volume or changes in the ventricular volume at that moment. For example, suppose two or more M-mode lines 12 are defined on a B-mode image 10 (as in FIG. 5B). If at least four non-collinear image locations 31, 32, 33, 34 are known in a transverse plane, a best-fit ellipse can be determined as an approximation to a transverse cross-section of the ventricle. (Mathematical techniques for fitting ellipses to known location coordinates are well known.) Then, one can compute estimates of the minimum and maximum radii of the ellipse, R₂ and R₃. Then Eq. 2a becomes the following:
V=(4π/3)·(R₁+C₁)·R₂·R₃+C2 Eq. 2b

Similarly, if at least four non-collinear image locations are known in a longitudinal image of the ventricle (such as shown in FIG. 3), then one can compute the radii R₁ and R₂ of a best-fit ellipse and therefrom the volume V of Eq. 2a or Eq. 2b.

If the locations of a sufficient number of sites on the cardiac wall are determined and the sites are adequately distributed three-dimensionally, then a closed surface geometrical model may be constructed. The location coordinates of cardiac wall sites may be obtained, for example, from the image planes of multiple ultrasound transducer arrays or from a 3-D ultrasound system as described herein. The multiple transducer arrays may be placed intravascularly, external to the heart, or both. If the 3-D coordinates of a dozen or so well distributed speckle points at a specific moment in cardiac cycle can be measured thereby, for instance, a polyhedron can be constructed with those points as its vertices to model a ventricle or atrium. The volume of such a polyhedron would approximate the volume of the modeled cardiac chamber, and the volume of the polyhedron is readily computed by known methods. Alternatively, a smoothly curved surface through the locations of cardiac wall sites (such as a NURBS surface) may be constructed using known computer graphics methods. The volume contained by a smoothly curved surface model may provide a better approximation of the volume of the cardiac chamber than a polyhedron model. The ejection volume for a cardiac chamber can thus be approximated by constructing such an approximating polyhedral or curved surface for cardiac wall sites measured at each of systole and diastole times in the cardiac cycle and calculating the difference between the volumes of those two surfaces.

An embodiment allows the user to define multiple M-mode cursor lines 12, as illustrated in FIG. 5B. Alternatively, an embodiment can be configured to automatically define M-mode cursor lines 12. For example, M-mode cursor lines 12 at some fixed angle (such as 10 degrees) on either side of a user-selected center M-mode line 12 may be defined. Further, image locations 31, 32, 33, 34 to be tracked on each M-mode line 12 may be automatically chosen (or suggested) based on echo amplitude thresholds, derivatives, and/or discontinuities (“edge detection” criteria) at cardiac wall boundaries. An embodiment can automatically search for and select an M-mode line 12 which exhibits maximum motion of discontinuities of amplitude along the line on an ultrasound (B-mode) image 10 plane.

Suppose that multiple M-mode lines 12 in multiple planes can be defined (manually or automatically) so that the coordinates of at least 6 non-coplanar sites can be tracked throughout at least one cardiac cycle. Then at any moment (phase) in the cardiac cycle, one can determine a best-fit ellipsoid, the ellipsoidal volume, and therefrom estimate the ventricular volume at that moment. In most cases, the estimates will be better when the 3-D points are more spatially separated from each other.

An embodiment automatically tracks small regional patterns or spots of speckle in the ventricular wall images from one frame to the next instead of or in addition to tracking points on selected M-mode cursor lines 12. For example, a speckle region may be a small circular or square region centered on an image location interactively chosen by the user of the system within the image of ventricular wall. As mentioned above, speckle is a feature of the “graininess” of an unprocessed ultrasound image and may be caused by very local constructive and destructive interference of the ultrasound waves and the reflections of those waves within tissue. Being particularly “bright” spots (i.e., being large amplitude echoes) in the ultrasound signal, speckle points can be readily selected by a user referring to a B-mode display or automatically selected by a system processor based upon the magnitude and dimensions of the reflected spot.

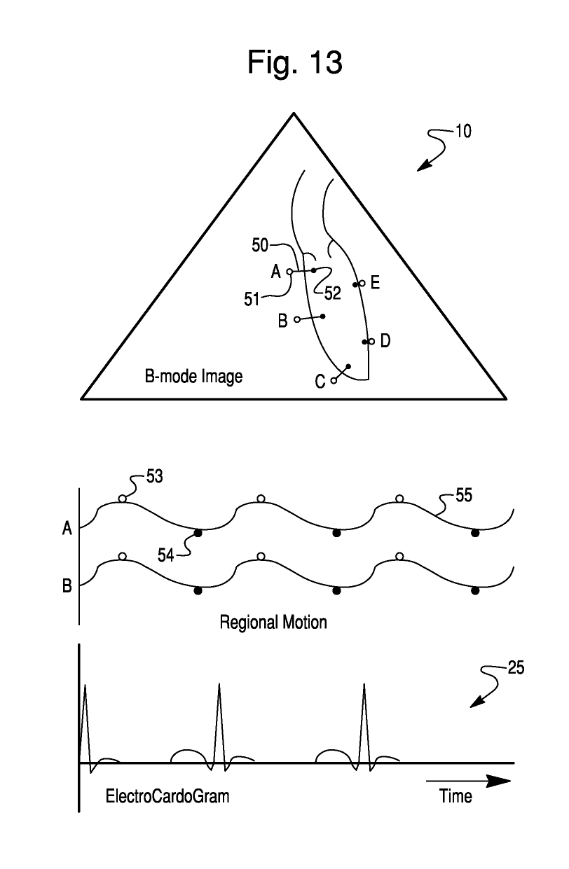

An M-mode image plots motions 41, 42 along a line (or lines)—which may be interactively chosen by the user by means of a moveable cursor line 12 on a B-mode image 10. Because a speckle pattern may move in a more arbitrary direction than along an M-mode cursor line 12, a variant display scheme may be necessary to display the relative timing (phase) of the motion of a speckle pattern. For example, a colored line 50 on a monochrome B-mode image 10 shown in FIG. 13 may trace the path of motion of the center of each user-specified speckle pattern location over the course of a cardiac cycle.

FIG. 13 illustrates an example of the paths 50 traced by five specified speckle regions A, B, C, D, and E. FIG. 13 also illustrates a way to plot the timing (phase) of the motion of regions A and B with respect to the cardiac cycle, which may be relative to an electrocardiogram signal 25, if available. A different color may be used for each motion path 50 on the image 10 (upper portion of FIG. 13) and the same color for the corresponding phase plot of the timing of the motion 55 of the regions (lower portion of FIG. 13). The phase plot of the motion of each region may be simply a single graph line 55. The small open circles 51, 53 and closed circles 52, 54 represent the two extremes of the motion paths 50 of the speckle regions. The relative timing (phase delay) of the motion 55 of a region from one extreme location to the other along its motion path may also be plotted (lower portion of FIG. 13). The phase of the motion of speckle regions may be displayed with respect to each other and/or to an electrocardiogram signal 25.

Volume can be calculated at systole and at diastole using any of various methods of estimating ventricular chamber volume. That is, volume may be determined with correlation to the ECG signal 25, as shown in FIG. 7A, by determining the minimum and maximum distances between image locations of the M-mode plots 41, 42, by the extrema locations 51,52 of the motion paths 50 of multiple speckle regions in the image of the ventricular wall (as in FIG. 13), or by measuring the distances at the onset of QRS complex, which is near the end of diastole, and at the beginning of the T wave which is near the end of systole based upon ECG signals. In any case, the stroke volume (ejection volume) is then given by:
V_{SV}=V_{diastole}−V_{systole} Eq. 3.

There will invariably be statistical variances in measurements of ventricle (or atrial) volume due to errors in measurements obtained from individual images (which may be due to both measurement error and imaging error) as well as variability in ventricle volume beat to beat. For this reason, multiple images and multiple measurements may be obtained to determine average volumes and the average ejection volume along with measures of the associated variances. As illustrated in FIG. 7D, a number of ultrasound images may be taken at points of maximum (img1) and minimum (img2) volume so the measurements may be averaged. While FIG. 7D shows two images in each state, a larger number N of images may be obtained in order to more accurately determine the average and variance of the measurements. Using this method, the ejection volume then can be determined by:

$${V}_{ejt} = \frac{{\sum }_{n = 1}^{N} {V}_{img 1}}{N} - \frac{{\sum }_{n = 1}^{N} {V}_{img 2}}{N} . \qquad Eq . 4$$

By obtaining multiple measurements of volume, an estimation of the error in volume measurements may be estimated using standard statistical analysis techniques.

One embodiment is in the form of hardware and/or software that exists as part of the ultrasound scanner (FIG. 1a). In such an embodiment, the system can utilize the Doppler processing capabilities of the host ultrasound scanner 4 to obtain a time-varying signal representative of the velocity of flow through an area of interest. Such an area could include the inlet of the aorta from the left ventricle, or the valve 13 in between (FIG. 3). The system also utilizes a view/measure of the cross-sectional area 14 through which the flow of interest is to pass (FIG. 5A).

The Doppler system outputs the spectral information 20, which is indicative of the velocity of flow through the volume of interest (as shown in FIG. 3) either by means of showing a spectrum (which in some embodiments can be obtained in analog or digital format from the machine). Such a spectrum can be obtained either by obtaining a longitudinal sectional view of the flow axis at any angle (as represented in FIG. 3), or by obtaining a cross sectional view of the flow conduit (FIG. 5). Such calculations of flow/area can compensate for the angle of measurement using a cosine of the angle with respect to actual plane correction. For conditions where the flow is perpendicular to the sample volume of the Doppler system, other estimation techniques such as “Transverse Doppler,” which utilizes the Doppler bandwidth to assess flow at flow to beam angles close to 90 degrees, can be utilized. See Tortoli et al., Ultrasound Med. Biol., 21, 527-532 (1995). This Doppler signal can also be as an audio signal (again, either in analog or digital format) as a frequency and/or amplitude modulated signal that is indicative of the spectrum and hence the flow velocity through the area of interest. This could further include ECG signals (again, in analog or digital format).

Because speckle tracking can be used to estimate the motion and distance that tissue has moved from one ultrasound video frame to the next (within the plane of the B-mode image), volumetric change can be measured and hence the displacement of blood may be computed. Therefore, speckle tracking can replace Doppler flow measurement within the imaged region, and Eq. 1a may be used to compute ejection volume. Specifically, the ejection fraction can be computed from the ejection volume Vₑⱼₜ and the estimate of ventricle's volume.

It is noted that using speckle points to track and measure sites on a ventricle wall may introduce some error if one or more speckle points shifts positions on the vessel with movement.

Further processing can be carried out, for example, using the following techniques for computing ejection volume based on flow velocity and flow cross-sectional area. In an embodiment, a user manually measures/demarcates, either with or without the aid of an ECG, one or more peak velocities on the spectrum and demarcates/measures the cross-section of the outlet of the ventricle. Once the peak velocities and cross section are entered, the system/calculating tool (either on the ultrasound machine or on a separate computer) then integrates the curve over time to obtain stroke volume via Eq. 1a.

Alternatively, a semi-automated process may be employed wherein the system (either on the ultrasound machine or separate) automatically integrates the curve with or without the help of an ECG input while the user inputs the area of interest of the orifice through which the flow passes.

Alternatively, a fully automated process may be employed wherein the system prompts the user to obtain particular views of the anatomy of interest and demarcate specific points and the system then processes the data as above with, however, the system internally tracking the data of interest.

In a further alternative embodiment, the system autonomously or semi-autonomously selects a number of speckle points on the ventricle, obtains multiple M-mode measurements using the selected speckle points and then processes the data as above to estimate stroke volume, ejection fraction, or other measure of ventricular function.

In an embodiment which tracks the image coordinates of N wall sites of the ventricular cavity, the ejection fraction may be estimated by determining a best-fit affine linear transformation M which maps the coordinates of the N sites at diastole to the coordinates of the same N sites at systole. That is, the transformation M approximately maps the coordinates of the N sites at diastole to the N sites at systole using uniform rotation, translation (lateral shift), and dimensional scaling (stretching/compression). Finding the best-fit M can employ, for example, the well-known least-squares technique, which in turn may use the singular value decomposition to determine M.
Di·M=Si approximately
where the multiplication is standard matrix multiplication

Di=coordinates of the ith point, at diastole, i=1, . . . , N

Si=coordinates of the ith point, at systole,

M=matrix form of the transformation.

The transformation M may be two-dimensional if the N sites are all in one plane or may be three-dimensional if the N sites can be obtained in multiple planes. The best-fit linear transformation M may be determined, for example, using the well-known least-squares method and may be represented by a homogeneous matrix M, as is common practice in computer graphics. Matrix M generally will have eigenvalues λⱼ, which essentially represent scaling factors, one for each of the dimensions, where there is some P such that P·M=λⱼ·P.

Then for the three-dimensional case, the ejection fraction Fₑⱼₜ may be approximated as the square root of the product of the eigenvalues of M^{T}M. For the two-dimensional case, the ejection fraction Fₑⱼₜ may be approximated by the product of the eigenvalues of M^{T}M, assuming that the longitudinal dimension of the cardiac cavity remains fairly constant. N must be at least 3 for non-collinear sites in a plane. N must be at least 4 for non-coplanar sites (the 3-dimensional case). Smaller inaccuracy will generally result for larger values of N and for wall sites which are distributed around the cardiac chamber.

The system can automatically integrate the curve from beat to beat, and output the stroke volume in any sort of display, having obtained the cross sectional area using the techniques mentioned above. Of course, various combinations and/or modifications of these techniques can be used if desired and depending on the particular application and/or patient.

In an embodiment which estimates the ejection volume or ejection fraction over a cardiac cycle, the estimate values can be enhanced by averaging the per-cycle stroke volume or the per-cycle ejection fraction over multiple cardiac cycles. Such averaging may provide a more representative mean value. Additionally, measurements over a number of cycles allow the computation of the statistical variation or standard deviation of the ejection volume or ejection fraction. The standard deviation then may provide an indication of the “error”, uncertainty, or untrustworthiness of the mean value. Also, the standard deviation may provide an indication of the beat-to-beat variability in cardiac output or efficiency.

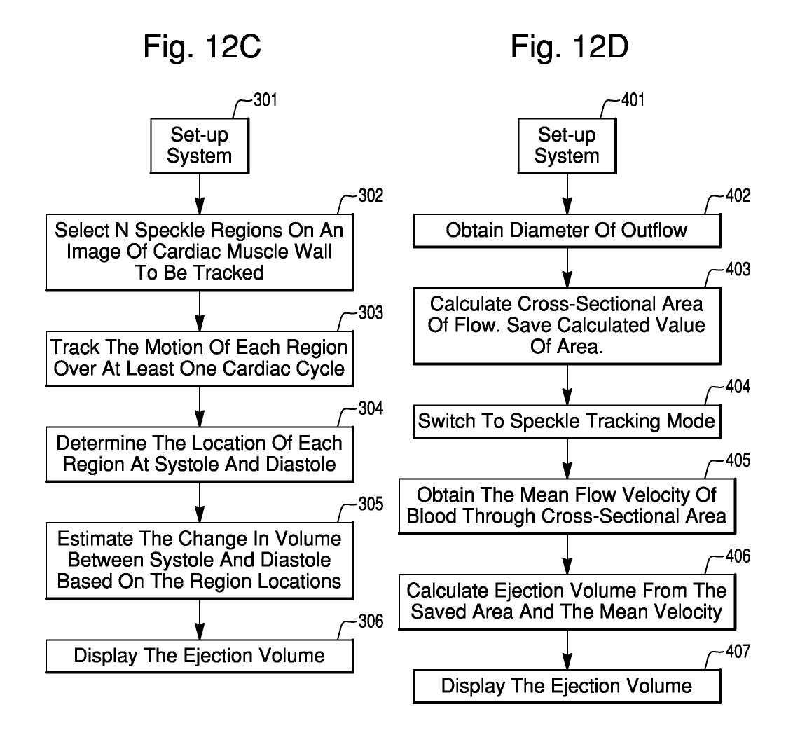

An example embodiment of the preceding processing using Doppler flow velocity measurements is provided as a flowchart in FIG. 12A. An example embodiment of the preceding processing using speckle tracking is provided as a flowchart in FIG. 12D.

In FIG. 12A, step 101 sets up and initializes the processing, and step 102 uses a B-mode image and the on-screen ultrasound system calipers to measure the diameter of the cross-sectional area of the cardiac flow in the image. Step 103 estimates and saves the area measurement based on the diameter. Step 104 switches the ultrasound operational mode to spectral Doppler. Step 105 selects at least three image locations at which to obtain velocity measurements, measures a velocity profile at those locations, and computes an average velocity. Alternatively, the average may be estimated as one-half the maximum velocity. Step 106 calculates the area containing the measured flow velocities. Step 107 retrieves the saved cross-sectional area, integrates the flow velocity over the time of a cardiac cycle, and multiplies by the cross-sectional area to obtain stroke volume. Step 108 displays the computed result as the ejection volume.

Referring to FIG. 12 D, a speckle tracking embodiment begins with step 401 which sets up and initializes the procedure. Step 402 measures the diameter of the cross-sectional area of the outflow using a B-mode image and the built-in calipers of the ultrasound system. Step 403 computes the area of the cross-section from the diameter and saves the value of the area. Step 404 switches the ultrasound system to speckle tracking mode. Step 405 uses speckle-tracking to estimate the mean flow velocity through the cross-sectional area of step 403. The mean flow may be estimated by dividing the maximum flow velocity by two or by sampling the flow velocities at three or more different image locations in the area and averaging the measurements of the velocities at those image locations. Step 406 integrates the average flow over the time of the cardiac cycle and multiplies by the area computed in step 403 to arrive at an estimate of the ejection volume. Step 407 displays the value of the ejection volume.

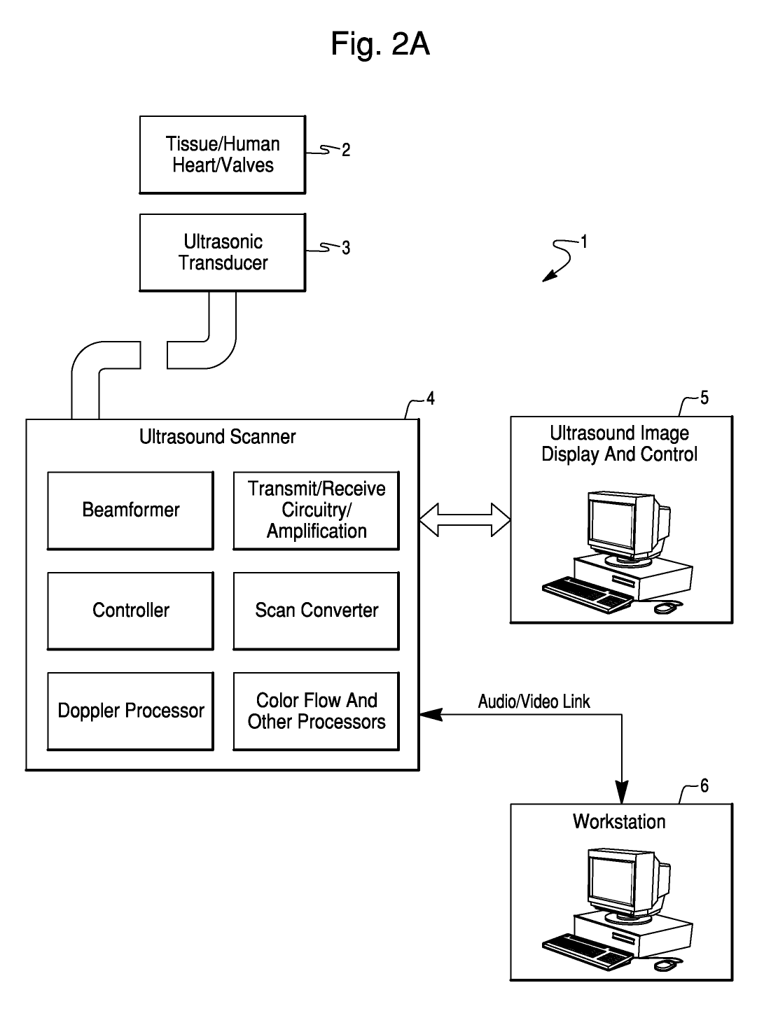

In another embodiment, the M-mode output is utilized to measure stroke volume. Again, this system can comprise hardware and/or software that resides wholly on the ultrasound scanner (FIG. 1) or can also include hardware and/or software on a separate workstation with means to communicate either digital and/or analog data with the ultrasound scanner (FIGS. 2A, 2B, and 2C). The volume can then be estimated, as given earlier by Equations 2 and 3.

Processing can be carried out, for example, using the following techniques for computing ejection volume based on the systolic and diastolic measurements. A user manually measures/demarcates, either with or without the aid of an ECG, the systolic and diastolic distances between the two ventricular walls, and the system/calculating tool (either on the ultrasound machine or on a separate computer) calculates the stroke volume. This process can include, if desired, provisions for the user or system to record/obtain the correction factors described in Equation 2.

Alternatively, a semi-automated process may be employed wherein the system (either on the ultrasound machine or separate) automatically measures the distances and estimates the stroke volume with or without the help of an ECG. In this case, the system can automatically measure/estimate the correction factors described in Equation 2, or the user can specify or aid the system in estimating/measuring these factors.

Alternatively, a fully automated process may be employed wherein the system prompts the user to obtain particular views of the anatomy of interest and demarcate specific image locations and the system then processes the data as above with, the system internally tracking the data of interest. The system can automatically measure the stroke volume, with data obtained from any of the above described methods, and output the stroke volume in any sort of display, having obtained the cross sectional area using the techniques mentioned above.

An example embodiment of the preceding processing is provided as a flowchart in FIG. 12B using M-mode measurements. An example embodiment of the preceding processing using speckle tracking is provided as a flowchart in FIG. 12C.

FIG. 12B uses M-mode based techniques. After step 201 sets up and initializes the processing, step 202 establishes a view showing two walls of the ventricle in M-mode. Step 203, using at least different three cross-sectional views of the ventricle, measures the differences between the maximum and minimum separations distances in each view using the built-in on-screen caliper tool of the ultrasound system. Step 204 estimates the change in ventricular volume from the at least three separation distances. Step 205 displays the resultant value of the estimated change in volume as the ejection volume.

In FIG. 12C, step 301 sets up and initializes the method. Step 302 selects some number N of small local regional speckle patterns within the image of the ventricular wall to track. Step 303 tracks the motion of each regional pattern over at least one cardiac cycle. Step 304 determines the change in location of each pattern. Step 304 may choose the locations of each pattern at systole and diastole, or step 304 may choose the two most widely separated locations (extrema) of the motion. Step 305 then estimates the change in ventricular volume from those locations. One way to make the estimate is to compute the volumetric difference between the best-fit ellipsoid matching the systole pattern locations and the best-fit ellipsoid matching the diastole pattern locations. Step 306 displays the estimated differential volume as the ejection volume.

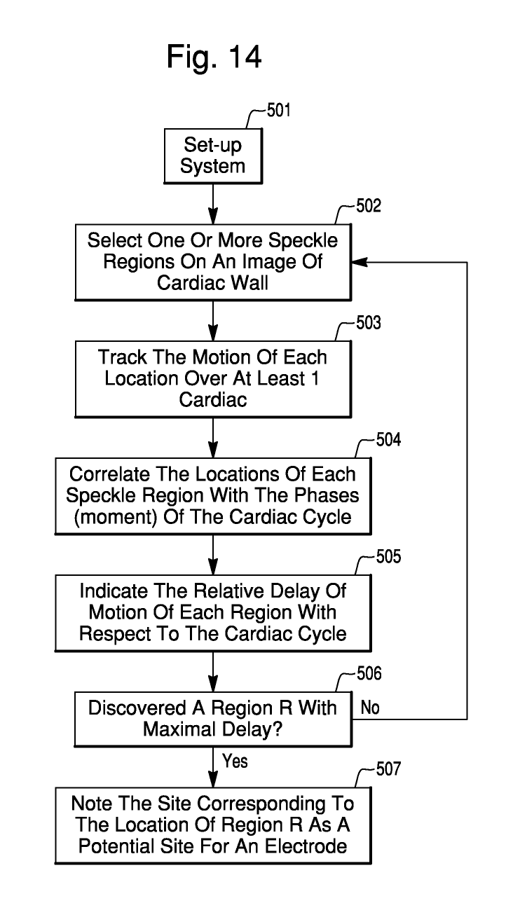

FIG. 14 provides a flowchart illustrating a procedure for determining a potential location for a pacemaker implant. Step 501 sets up and initializes the procedure. Step 502 selects at least one location on the image of the ventricular wall to be tracked. That image location corresponds to a selected cardiac site. Using speckle pattern tracking techniques, step 503 tracks the motion of each selected site during at least one cardiac cycle by tracking the location of the image of the site. It may be assumed for these purposes that a speckle pattern generally follows the image location of a corresponding cardiac site. Step 504 correlates the relative motion of the image of each site with respect to the cardiac cycle. For example, the motion of a speckle pattern may be tracked as a fraction of the distance along the motion path from one end or extreme of the path to the other end or extreme. The motion may be plotted over the time or phase of a cardiac cycle as a line 55 for each tracked regional site on the cardiac wall as exemplified in FIG. 13. The phase delay of the site's motion relative to an ECG signal (if available) or relative to the motion of another site may be displayed graphically by step 505 and observed. Alternatively, a correlation computation may compute and display a numerical value for the phase difference between the motions of two sites or between the motion of each site and the ECG signal. The above steps may be repeated some number of times for multiple electrode sites on the cardiac wall until a site is found with maximal phase lag behind the ECG signal (if present) or behind the motions of other sites. Step 507 then suggests such a site as a potential site for a stimulation electrode. Thereafter, with the electrode in operation, any of the methods presented above for estimating ejection volume may be used to verify or measure improvement in the ejection volume. Further, measurements of the phase delay of the site's motion relative to an ECG signal (if available) or relative to the motion of another site may be used to set a parameter of the pacemaker, such as the pacing timing lead or delay for the electrode attached at the site.

Additionally, imaging and site motion tracking according to various embodiments may be used to image an unsteady pacing area or electrically malfunctioning area within the heart detected or located using ECG sensor data. For example, U.S. Patent Provisional Application No. 60/795,912, which is incorporated herein by reference in its entirety, describes methods for locating malfunctioning areas of the heart using ECG data mapped on an anatomical model of the heart. By using the various embodiments of the present invention to track or image the unsteady pace or otherwise malfunctioning region in the conductive pathway, the resulting site tracking or images may enable the physician to more accurately locate and optimize the positions for pacing leads. Further, motion tracking of the selected region may enable the physician to more accurately optimize the pacing timing and rhythm for the lead, both by measuring the lag before emplacement to estimate an appropriate timing parameter and by measuring the lag after pacing is initiated to confirm the region is responding as desired to the pacing stimulation. Additionally, site tracking and ultrasound images of the heart may be used to correct, correlate or otherwise improve the anatomical model used for displaying ECG data.

Another embodiment of the present invention is in the form of hardware and/or software that exists separate from the ultrasound scanner console or workstation with means to communicate either video and/or audio and/or other signals between the ultrasound scanner and/or the display computer/system (as exemplified in FIGS. 2A, 2B, and 2C). Communication between such workstation and the ultrasound scanner could include video, audio, and/or any ECG signals in digital and/or analog format. The above described processing can then be performed either partially or entirely on the workstation.

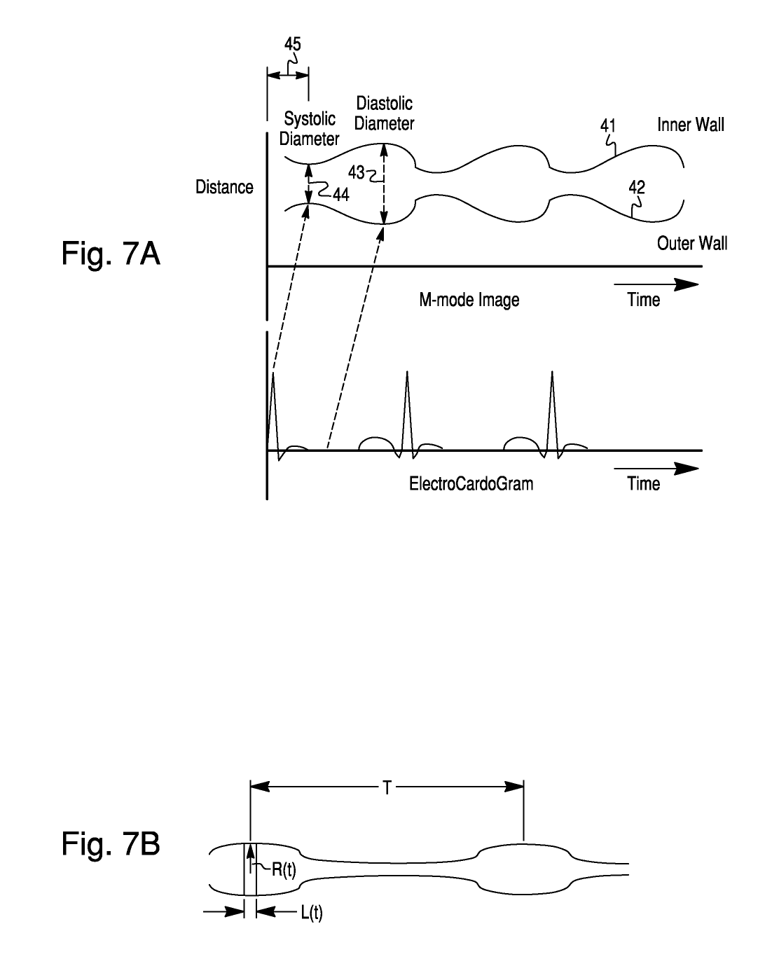

Another embodiment can include hardware and/or software separate from the ultrasound scanner, in the form of a workstation 6 wherein there exists a mode of communication, either analog or digital, between the workstation 6 and the ultrasound scanner 4 or catheter 3. See FIGS. 2A and 2B. Cabling from the ultrasound machine 4 to the catheter 3 (especially with a multi element array catheter) and from the catheter proximal connector to the catheter transducer housed at the distal tip can be expensive. To reduce cost, the ultrasound machine 4 can be moved adjacent to the patient 2, thereby allowing a relatively short cable to be used to attach the catheter. In some cases, however, this may be impractical since most catheter rooms are sterile or semi-sterile environments and, thus, the ultrasound machine may be some distance from the patient's bedside. Thus, a connecting cable which is reusable (and probably non-sterile) is desirable, as opposed to the catheter itself, which is sterile and usually not re-usable. It would be desirable if this connecting cable could be used as a universal cable in that it could be used with many ultrasound machines. While many ultrasound machines have a standard 200 pin ZIP connector, most ultrasound machines do not have patient isolation means built in to the degree necessary for percutaneous catheter use. Therefore, in another embodiment as shown in FIG. 8, the system of this invention employs a connector cable with an isolation box 7 that is external to the ultrasound machine 4 itself. Preferably the isolation box, which may house a plurality of isolation transformers, is relatively small so that it can be placed easily on or near the patient's bed. Such a cable can accommodate all operational communication between the catheter 3 and the ultrasound machine and/or the appropriate computer workstation.

In still another embodiment, the ultrasonic catheter further comprises a temperature sensing and/or control system. Especially when used at higher power (e.g., when using color Doppler imaging) and/or for lengthy periods of time, it is possible that the transducer, and hence, the catheter tip, generate heat that may damage tissue. While computer software can be used to regulate the amount of power put into the catheter to keep the temperature within acceptable ranges, it is also desirable to provide a temperature sensing means as well as a safety warning and/or cut-off mechanism for an additional margin of safety. Actual temperature monitoring of the catheter tip is most desirable, with feedback to the computer, with an automatic warning or shut down based upon some predetermined upper temperature limit. The system can be programmed to provide a warning as the temperature increases (e.g., reaches 40° C. or higher) and then shut off power at some upper limit (e.g., 43° C. as set out in U.S. FDA safety guidelines). To monitor the temperature at or near the tip of the catheter (i.e., in the region of the ultrasound transducer), a thermistor may be used. The temperature at the tip of the catheter can be continuously monitored via appropriate software. Although the software can also provide the means to control the power to the catheter in the event that excessive temperatures are generated, it may also be desirable to have a back up, shut off, or trip mechanism (e.g., a mechanical or electrical shut off or tripping circuit).

In a further embodiment, the ultrasound system, isolation box and temperature monitoring/cutoff circuits may be packaged as a combined unit which can be placed close to the patient and eliminate or shorten some of the cables required for a system comprised of separate components.

Of course, various combinations and/or modifications of these techniques and systems can be used if desired and depending on the particular application and/or patient.

It is to be understood, however, that even though numerous characteristics and advantages of the present invention have been set forth in the foregoing description, along with details of the structure and function of the invention, the disclosure is only for illustrative purposes. Changes may be made in detail, especially in matters of shape, size, arrangement, storage/communication formats and the order of method steps within the principles of the invention to the full extent indicated by the broad general meaning of the terms in which the appended claims are expressed.

What is claimed is:

1. An ultrasound imaging system suitable for evaluating a patient's heart, comprising:
an ultrasound imaging catheter including at least one ultrasound transducer;
ultrasound processing electronics coupled to the ultrasound imaging catheter and configured to process electrical signals from the at least one ultrasound transducer to generate ultrasound image signals; and
a computer coupled to the ultrasound processing electronics and configured to process the ultrasound image signals to measure a volume of a chamber of the heart using speckle tracking.

2. The ultrasound imaging system of claim 1, wherein the computer is further configured to estimate ventricular ejection volume using speckle tracking.

3. The ultrasound imaging system of claim 1, wherein the computer is configured to determine a delay measurements at various sites on the heart using speckle tracking.

4. The ultrasound imaging system of claim 2, wherein the computer is configured to autonomously select speckle points, measure movement of the selected speckle points and estimate the ejection fraction based upon the measured movement of the selected speckle points.

5. The ultrasound imaging system according to claim 4, wherein the computer is further configured to estimate the ejection volume of the patient's heart over a plurality of cardiac cycles and output an average ejection volume per cardiac cycle.

6. The ultrasound imaging system according to claim 5, wherein the computer is further configured to compute a standard deviation of the ejection volume over the plurality of cardiac cycles.

7. An ultrasound imaging system suitable for measuring cardiac output of a patient's heart, comprising:
an ultrasound imaging catheter including at least one ultrasound transducer;
an ultrasound processor coupled to the ultrasound imaging catheter, the ultrasound processor configured to receive signals from the at least one transducer and output speckle pattern tracking data; and
a computer configured to receive the speckle pattern tracking data and use the speckle pattern tracking data to compute a measure of cardiac output of the patient's heart.

8. The ultrasound imaging system according to claim 7, wherein the ultrasound processor is further configured to choose speckle corresponding to sites on a ventricle wall and measure motions of the sites using speckle tracking.

9. The ultrasound imaging system according to claim 7, wherein the computer is further configured to estimate a volume of a ventricle of the patient's heart.

10. The ultrasound imaging system according to claim 9, wherein the computer is further configured to estimate a maximum and a minimum volume of the ventricle and to compute a difference in maximum and minimum volumes as the measure of the cardiac output of the patient's heart.

11. The ultrasound imaging system according to claim 11, wherein:
the ultrasound processor is further configured to output a measure of blood flow across a cross-sectional area obtained from a transverse ultrasound image of a ventricle; and
the computer is further configured to estimate blood flow velocity using speckle pattern tracking data obtained from a longitudinal image of a ventricle and the output blood flow measurement.

12. The ultrasound imaging system according to claim 11, wherein the computer is further configured to compute cardiac output of the patient's heart over a plurality of cardiac cycles and compute an average measure of cardiac output per cardiac cycle.

13. The ultrasound imaging system according to claim 12, wherein the computer is further configured to compute a standard deviation of the cardiac output over the plurality of cardiac cycles.

14. A method for measuring cardiac output of a patient's heart, comprising:
imaging a portion of the patient's heart using an ultrasound imaging catheter which includes at least one transducer;
obtaining dimensional measurements of at least 6 speckle spots corresponding to non-coplanar sites on a ventricular wall at a systole phase;
obtaining dimensional measurements of the at least 6 speckle spots at a diastole phase;
determining a first volume of a best-fit systole ellipsoid using the obtained dimensional measurements of the at least 6 speckle spots at the systole phase;
determining a second volume of a best-fit diastole ellipsoid using the obtained dimensional measurements of the at least 6 sites at the diastole phase;
computing a difference between the first and second volumes as the measure of cardiac output of the patient's heart.

15. The method of claim 16, further comprising autonomously selecting the at least 6 speckle spots corresponding to sites on the ventricle wall.

16. A method for placing cardiac pacing electrodes within a patient's heart, comprising:
positioning an ultrasound imaging catheter near or within the patient's heart;
obtaining an ultrasound image of a portion of a ventricle using the ultrasound imaging catheter for at least one cardiac cycle;
selecting at least one speckle spot corresponding to a site on a wall of the ventricle within the ultrasound image of the ventricle;
tracking motion of the at least one speckle spot for at least one cardiac cycle;
determining a phase delay of the motion of the at least one speckle spot relative to a systole phase of the cardiac cycle;
repeating the electing, tracking and determining steps for another speckle spot corresponding to another site on the wall of the ventricle until a speckle spot is discovered which has a maximal phase delay; and
placing the cardiac pacing electrode at the discovered speckle spot with maximal phase delay.

17. The method of claim 16, further comprising setting a parameter of a pacemaker based upon an amount of phase delay of the discovered speckle spot.

18. The method of claim 16, wherein:
tracking motion of the at least one speckle spot is performed over a plurality of cycles; and
determining the phase delay comprises determining a mean phase delay over the plurality of cycles.

19. The method of claim 18, further comprising computing a standard deviation of the phase delays over the plurality of cardiac cycles.

20. The method of claim 16, wherein determining the phase delay of the motion of the at least one speckle spot is relative to an R wave of an electrocardiogram signal.
